(19) United States
(12) Patent Application Publication
(10) Pub. No.: US 2019/0231233 A1
(43) Pub. Date: Aug. 1, 2019

(54) HEARING TEST AND MODIFICATION OF AUDIO SIGNALS

(71) Applicant: GOSHAWK COMMUNICATIONS LIMITED, Douglas (GB)

(72) Inventors: Matthew TURNER, Knebworth (GB); Brian MOORE, Cambridge (GB); Michael STONE, Chapel-en-le-Frith (GB)

(21) Appl. No.: 16/315,490

(22) PCT Filed: Jul. 7, 2017
(86) PCT No.: PCT/EP2017/067168

§ 371 Date: Jan. 4, 2019

(30) Foreign Application Priority Data
Jul. 7, 2016 (GB) 1611804.4

(51) Int. Cl. A61B 5/12; A61B 5/00; G06F 3/16

(52) U.S. Cl. CPC A61B 5/125; A61B 5/0022; A61B 2560/0247; G06F 3/165; A61B 5/6898

(57) ABSTRACT

A method comprising: conducting a hearing test for a user over a communication link established between a network entity in a communication network and a user device of a user; wherein the hearing test comprises providing audio stimuli to the user device at a plurality of test frequencies over the communication link, and monitoring responsiveness to the audio stimuli received from the user device; generating a hearing profile based on results of the hearing test; and storing the hearing profile and information associated with the user in a memory of a network entity, such that the hearing profile is available for modifying of audio signals to the user device.

### FIELD

This disclosure relates to a hearing test. This disclosure also relates to the modification of audio signals, for example speech and music, using results of the hearing test. It is particularly suitable for, but by no means limited to, enhancement of audio signals for people with addressable hearing loss or needs, in particular over a communications network such as a mobile telephone network.

### BACKGROUND

The current solutions for enhanced audio over a mobile or fixed device, for example a mobile or landline phone, provide software applications that can be loaded into or implemented by typical user devices to simulate a hearing aid on a mobile or fixed terminal, for example by making use of digital technology to use local processing at the user device to emulate a hearing aid for people with mild to severe hearing loss, but not for the case of profound to extreme hearing loss that may require specialist treatment or medical solution. Other solutions provide complex device accessories as add-ons to a mobile device by way of replacing or working in combination with a hearing aid or implant for people with mild to severe hearing loss.

Such solutions require processing power at the user device and/or additional hardware or firmware.

Accordingly, there is a need for providing the convenience of audio enhancement carried out by a central system, for example at the network level, such that the enhancement is transparent to a user device and can therefore be implemented or provided on or to any user device (which may be mobile, fixed or a stand alone speaker or other such communication method), and not restricted to higher end devices with greater processing power and local resources. Further, avoiding the need for device accessories may increases audio enhancement availability for more users as hardware or firmware requirements are reduced, implementation costs and energy use may be lower, hence potentially allowing audio enhancement to reach a wider range of users.

### SUMMARY

According to an aspect, there is provided a method comprising: conducting a hearing test for a user over a communication link established between a network entity in a communication network and a user device of a user; wherein the hearing test comprises providing audio stimuli to the user device at a plurality of test frequencies over the communication link, and monitoring responsiveness to the audio stimuli received from the user device; generating a hearing profile based on results of the hearing test; and storing the hearing profile and information associated with the user in a memory of a network entity, such that the hearing profile is available for modifying of audio signals to the user device.

The information associated with the user may comprise an identifier of the user and/or an identifier of the user device.

According to some embodiments the network entity in which the hearing profile is stored is the same network entity which has the communication link with the user device.

According to some embodiments the network entity in which the hearing profile is stored comprises a second network entity, and the network entity which has the communication link with the user device comprises a first network entity, the first and second network entities being in communication with each other.

According to some embodiments the identifier comprises a unique identifier.

According to some embodiments, the identifier comprises an MSISDN.

The audio stimuli may comprise white noise, the white noise based on one or more human voices.

The audio stimuli may comprising ⅓ octave wide bands of noise.

The providing of audio stimuli to the user at a plurality of test frequencies may comprise providing audio stimuli at two or more of 500 Hz; 1000 Hz; 2000 Hz; 3000 Hz; 6000 Hz.

According to some embodiments, the plurality of test frequencies are provided to the user in a step-wise fashion.

According to some embodiments, the method comprises synchronising clocks between the user device and the network entity which has the communication link with the user device prior to playing the audio stimuli.

The method may comprise obtaining an indication of hearing loss of the user, and using the indication of hearing loss to determine an initial volume of the hearing test.

The method may comprise adjusting a volume of the audio stimuli at each test frequency in response to the monitoring responsiveness.

In response to a positive response from the user the method may comprise decreasing the volume of the audio stimuli.

According to some embodiments, the decreasing the volume comprising decreasing the volume in 5 dB steps.

In response to a null response from the user, the method may comprise increasing the volume of the audio stimuli.

According to some embodiments, the increasing the volume comprises increasing the volume in 10 dB steps.

A duration of each audio stimuli may be at or about 1000 ms.

Each audio stimuli may comprise one or more ramps of increasing/decreasing volume between a background noise level and 60 dB or about 60 dB.

The method may comprise visually displaying results of the hearing test to the user and/or an operator.

The method may comprise using the stored hearing profile of the user to modify audio signals to the user in real-time, the modifying of the audio signals being carried out at the network entity such that modified audio signals are delivered to the user device of the user.

The modifying audio signals may comprise one or more of: filtering the audio signal; adjusting the amplitude of the audio signal; adjusting the frequency of the audio signal; adjusting the pitch and/or tone of the audio signal.

According to some embodiments the audio signal modification is executed by a sound processing engine comprising a network interface.

The modifying audio signals may comprise modifying voice signals of a second user in a call between the user and second user.

The method may comprise: enabling selective activation or deactivation of a setting which provides the audio signal modification.

The method may comprise measuring ambient noise using one or more microphones of the user device, receiving ambient noise information from the user device at the network entity that has the communication link with the user device, and storing the received ambient noise information at the network entity which stores the hearing profile for use in modification of audio signals to the user.

The method may comprise determining a channel insertion gain for delivering the audio signals to the user device.

According to some embodiments, the determined channel insertion gain is user-specific.

According to some embodiments, the determining a channel insertion gain comprises dynamically varying the gain.

The method may comprise splitting the audio signals in to multiple channels.

According to some embodiments the multiple channels comprises three or four channels.

The method may comprise determining a power level for each channel.

According to some embodiments, the determining a channel insertion gain comprises using user parameters.

According to some embodiments the user parameters comprise one or more of: an initial perceived estimate of the user hearing threshold; an initial user volume preference; an audiogram or a combined digital hearing threshold information of a user based on the combined input parameters of the user hearing loss and device in use to generate such a hearing threshold; age of a user; hearing aid information of a user; gender of user.

The channel insertion gain may be applied prior to dynamic compression of the audio signals to the user.

According to some embodiments the dynamic compression comprises determining attack and release levels for each channel.

According to some embodiments the attack level comprises a time for a gain signal to settle relative to a final value, and the release level comprises a time for the gain signal to settle relative to a final value.

According to some embodiments the attack level comprises a time for a gain signal to settle within 3 dB of a final value, and the release level comprises a time for the gain signal to settle to within 4 dB of a final value, for a 35 dB change applied at a compressor for the dynamic compression.

According to some embodiments, the method comprises processing audio signal frames prior to transmission of the audio signal frames to the user, the processing of the audio signal frames comprising applying a finite impulse response filter to the audio signal frames.

Some embodiments may comprise a server arranged to carry out the method of any of the method features described previously.

According to another aspect, there is provided a method comprising: participating in a hearing test for a user over a communication link established between a user device and a network entity in a communications network to provide a hearing profile for a user; wherein the hearing test comprises receiving audio stimuli at the user device at a plurality of test frequencies over the communication link, and providing one or more responses to the audio stimuli to the network entity; and subsequently receiving audio signals at the user device modified in dependence on the hearing profile.

Some embodiments may comprise a user device arranged to carry out this method.

According to an aspect there is provided a user device comprising a display, and a plurality of microphones. According to some embodiments the plurality of microphones are directionally focused.

According to some embodiments the microphones are configured for communication with an operating system of the user device.

According to some embodiments the microphones are configured to detect ambient noise.

According to some embodiments the user device is configured to provide information of the ambient noise to a network entity.

According to some embodiments the user device comprises a coating or layer.

According to some embodiments the coating or layer is configured to act as an antenna and/or an induction loop and/or a tele-coil.

According to some embodiments the coating or layer comprises a battery and/or a processor and/or a memory.

According to some embodiments the coating or layer comprises tagging and/or internet of things capability.

According to some embodiments the coating or layer is in the form of a casing which is attachable and detachable from the user device.

According to some embodiments the user device may be used in conjunction with the methods described herein.

According to another aspect there is provided a method of real-time enhancement of an audio signal to a first user. This may provide a real-time enhancement without undue delay. Thus there is provided a method of real-time enhancement of an audio signal to a first user on a network comprising characterising a first user's hearing in a unique hearing profile, the profile comprising predetermined parameters, the parameters being derived from hearing capabilities of the first user at predetermined input frequencies and using the predetermined parameters of the hearing profile to enhance the audio signal to the first user in real time.

Optionally, enhancing the audio signal comprises filtering originating audio signal and/or adjusting amplitude and/or frequency according to the predetermined parameters of the first user's hearing profile.

Optionally, the method further comprises i characterising a second user's voice in a unique voice profile, the profile comprising predetermined parameters, the parameters being derived from voice pitch and/or tone of the second user and using the predetermined parameters of the voice profile to enhance the audio signal to the first user in real time.

Optionally, enhancing the audio signal comprises shifting the pitch and/or tone of the second user's voice according to the second user's voice profile towards requirements defined by the first user's hearing profile.

Optionally, the method further comprises characterising the ambient noise of the network in an ambient noise profile, the profile comprising predetermined ambient noise parameters and using the predetermined ambient noise parameters to enhance the audio signal to the first user in real time.

Optionally, the predetermined ambient noise parameters comprise at least one of signal to noise ratio, echo, device transducer effect or data packet loss.

Optionally, the audio signal enhancement is executed by a sound processing engine comprising a network independent interface.

Optionally, the network independent interface comprises a first interface with a parameter database and a second interface with an audio signal data packet interface for intercepting and enhancing the audio signal in real time.

Optionally, the second interface comprises an RTP interface.

Optionally, the sound processing engine resides on a server and the enhanced audio signal is delivered to the first user's device pre-enhanced.

Optionally, the sound processing engine resides on the first user's device and the enhanced audio signal is provided to the first user after the sound processing engine has received the predetermined parameters.

Optionally, the audio signal is carried in audio data packets on an IP network and further wherein the audio data packets are routed to the sound processing engine by way of SIP via a media gateway.

Optionally, hearing profile parameters are derived by testing a user's hearing at the predetermined frequencies with white noise based on one or more human voices.

Optionally, each user is identified by a unique identification reference.

Optionally, enhancement of the audio signal is capable of being enabled and disabled in real time.

Optionally, the parameters of the hearing profile are determined after synchronisation of user device and server clocks respectively.

Optionally, the parameters of the hearing profile are changed based on at least one of age of the user, sex of the user, or time since last hearing profile parameters were derived.

Optionally, a voice profile is associated with a user unique identification reference such as an MSISDN such that re-characterisation of a user's voice in a voice profile is not required when the user is using the known MSISDN.

According to another aspect there is provided a user device comprising a processor arranged to perform the above method.

According to another aspect there is provided a server arranged to carry out the above method (s).

According to another aspect, there is provided a computer program product for a computer device, comprising software code portions for performing the steps of any of the above method aspects, when the program is run on the computer device. The computer device may be a server, a computer, a user device, a mobile phone, a smart phone or any other suitable device.

According to another aspect there is provided a computer readable medium comprising instructions that when executed, cause a processor to carry out any of the previous methods.

A computer program comprising program code configured when run on at least one processor to cause any of the previous methods to be performed.

In the above, many different embodiments have been described. It should be appreciated that further embodiments may be provided by the combination of any two or more of the embodiments described above.

### BRIEF DESCRIPTION OF THE DRAWINGS

Embodiments will now be described, by way of example only, and with reference to the drawings in which:

FIG. 1 illustrates an architectural overview of two users communicating via enhanced audio as provided in an embodiment;

FIG. 2 illustrates a high level example of a call initiated over a PSTN as well switching and routing of the calls providing a voice enhancement service according to an embodiment;

FIG. 3 illustrates data protocol flow involving when audio enhancement is taking place according to an embodiment;

FIG. 4 illustrates the audio enhancement component deployed in relation to first/second networks according to an embodiment;

FIG. 5 illustrates data flow associated with call initiation and audio enhancement by the sound processing engine according to an embodiment;

FIG. 6 illustrates the processes involved in acquiring a user's hearing and voice profile by way of input conditioning (FIG. 6A), output conditioning (FIG. 6B) and ambient conditioning (FIG. 6C) according to an embodiment;

FIG. 7 illustrates processing steps undertaken by the sound processing engine when it is enhancing audio according to an embodiment;

FIG. 8 illustrates frequency response of the audio enhancement;

FIG. 9 illustrates the frequency spectrum of real time audio enhancement using wideband voice processing at 16 kHz;

FIG. 10 illustrates the frequency spectrum of real time audio enhancement using narrowband voice processing at 8 kHz;

FIG. 11 illustrates an example user device according to an embodiment;

FIG. 12 illustrates a flow chart of a method according to an example;

FIG. 13 illustrates a flow chart of a method according to an example; and

FIG. 14 illustrates a user device according to an example.

In the figures, like elements are indicated by like reference numerals throughout.

### DETAILED DESCRIPTION

Overview

This disclosure illustrates a hearing test and audio enhancement of voice signals, in particular over a communications network, for example a mobile communications network. This disclosure utilises an approach whereby parameters associated with a user are first assumed on a pre-defined basis and subsequently refined in the hearing test and then used to enhance the audio associated with that user, preferably centrally, whenever that user is communicating over the communications network. The parameters associated with any user's hearing characteristics are referred to as their hearing biometrics and may be protected by way of encryption in the network to avoid unwarranted access to that information.

That is to say that a central communications network provides fixed or mobile access to audio enhancement, for example via a cloud service, or other central resource. Hence, the enhanced audio signal can be provided by way of any central resource accessible to both users and with which at least one of the users has registered voice and/or hearing parameters in the form of a profile, such that those parameters can be applied to the audio signal to provide a unique enhanced signal, tailored for that user (originating from and/or being delivered to the user), preferably centrally, or optionally at that user's device.

Architecture

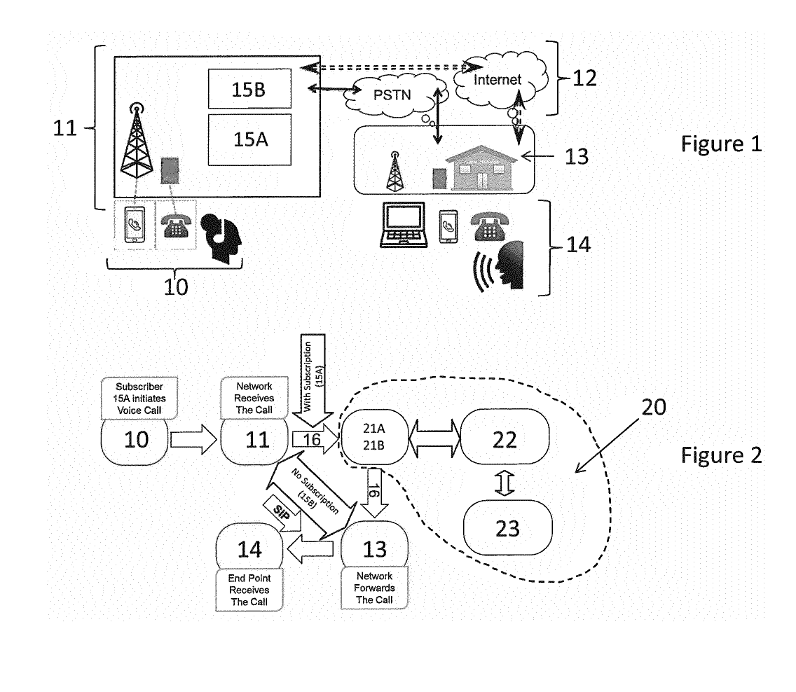

Turning to FIG. 1, an architectural overview is shown of two users communicating via enhanced audio as provided in an embodiment. A first user 10 with a communications device connected to a first network 11 and a second user 14 with a communications device connected to a second network 13 are able to communicate via communication means 12. The first and second networks may comprise any of a mobile communications network, a fixed line network or a VoIP network. Communication means 12 may comprise a PSTN, the internet, WAN LAN, satellite or any form of transport and switching network capable of delivering telecommunication services, for example but not limited to fixed line, WiFi, IP networks, PBX (private exchanges), apps, edge computing, femotocells, VoIP, VoLTE, and/or Internet of Things. Basically, any means by which a digital or analogue signal can be transmitted/distributed such as a national or local power distribution network (the National Grid in the UK) and capable of delivering an audio signal to a user end device which then processes the signal including audio enhancement. In other embodiments, audio enhancement may be processed on the user device as an app or embedded firmware.

In FIG. 1, first user 10 may be a subscriber 15A to the disclosed enhanced audio service or a non-subscriber 15B. A subscriber 15A is able to gain access to enhanced audio processing by way of audio enhancement component 20 as described further herein.

Based on the architectural structure shown in FIG. 1, and turning to FIG. 2, a high level example of a call initiated by first user 10 over a PSTN 12 operates as now described. Once a call is initiated, first network 11 detects whether the first user 10 is a subscriber 15A. If so, audio enhancing is provided by way of audio enhancement component 20, if not, a standard call is forwarded by first network 11 to second user 14 via PSTN 12.

Audio enhancement component 20 (shown by way of the area inside the dashed line) comprises a media gateway controller 21A, media gateway 21B, sound processing engine 22 and configuration management module 23, and may be positioned within the core network of a communication network, in this embodiment the first network 11. In the embodiment of FIG. 2, session initiation protocol (SIP) 16 is used to initiate a call as would be understood (and allow creation of additional audio enhancement services) involving audio enhancement via media gateway 21B of audio enhancement component 20. Other appropriate non-IP protocols may alternatively be used. The embodiments described herein may utilise standard network interfacing components and protocols such as IP, SIP and VoIP protocols and various components such as a session border controller (SBC) or a media gateway and its controller or equivalent to connect with telecommunication or other underlying networks. Such networks may vary in their signalling and interfaces based on today's technology for legacy CAMEL/IN, ISDN or IMS network specifications when communicating with fixed or mobile networks as would be understood.

As would be understood, networks 11, 13 may vary based on the ‘last mile’ access and core network technology used for connecting to their users. Media gateway 21B provides means for conversion of signalling as well as traffic from a variety of possible standards from, for example, legacy operator networks to more recent IP based solutions. SIP for signalling and RTP for traffic flow of a voice service.

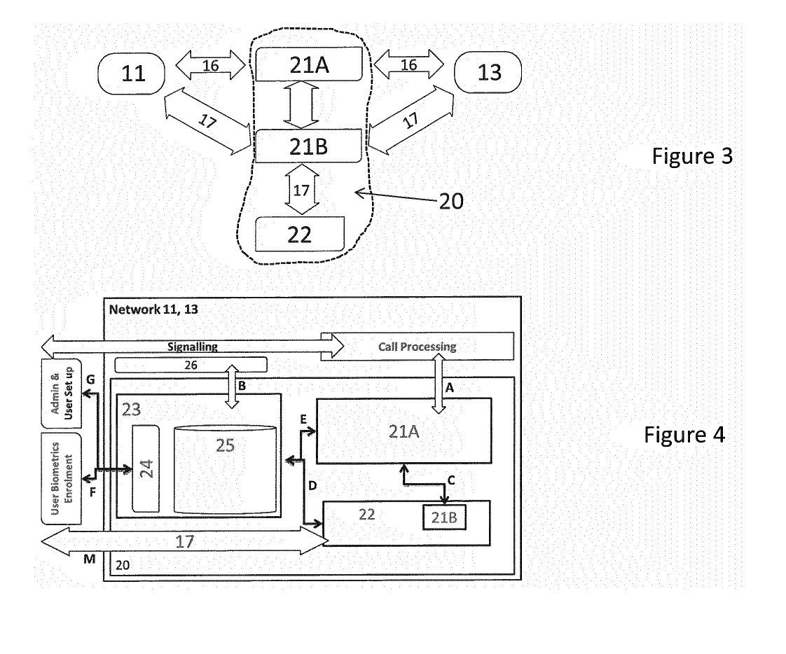

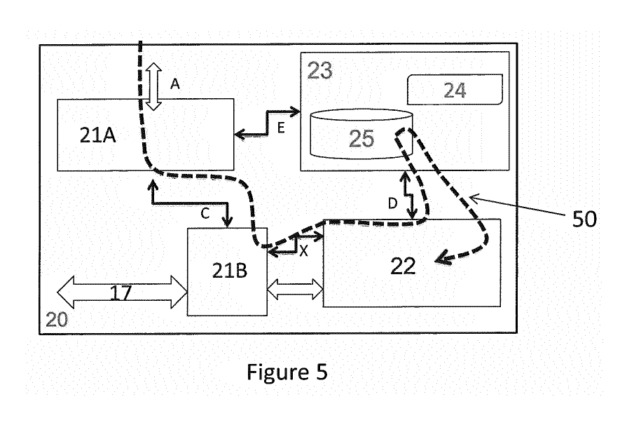

Before audio enhancement component 20 is described in more detail, FIG. 3 illustrates data protocol flow involving audio enhancement component 20 when audio enhancement is taking place on the underlying architecture of FIG. 1. Media gateway controller 21A deals with initiation of an enhanced audio call (in this embodiment by way of SIP packets). Media gateway 21B deals with multimedia real time protocol (RTP) packets 17 including an interface with sound processing engine 22 (see interfaces ‘D’ and ‘X’ described herein) and is in communication between first network 11 to/from first user 10 and second network 13 to/from second user 14 of an on-going call as would be understood. Sound processing engine 22 modifies the audio stream contained in the RTP packets 17 originating from and/or provided to first user 10 subsequent to SIP 16 initiation such that first user 10 (in the embodiment of FIG. 1 and who is a subscriber 15A to enhanced audio processing) is provided with audio enhancement based on a hearing and voice profile contained within configuration management module 23. Sound processing engine may additionally be capable of using a different hearing and voice profile in either direction such that two users with hearing impairment may have their audio enhanced simultaneously (see FIG. 5 and accompanying text).

As described later, in an alternative embodiment, interfaces ‘D’ and ‘X’ allow sound processing engine 22 to reside at a distributed node of a network, for example associated with a mobile network of any country or in a user device by way of a pre-installed codec, for example, if the user device has enough processing power and local resources. In such an embodiment, configuration management module 23 provides parameters to be utilised by the codec when providing audio enhancement. Accordingly, hearing biometric data centrally may be kept within the network, and it is possible to execute the sound enhancement function as a distributed functional node in a server operating physically in a location other than where configuration management system 23 is executed or the media gateway 21B is operating. This distributed functionality of the sound enhancement can be considered to be executed at the edge of the network closer to the user's (10, 14) device, or in certain cases where compatibility and interoperability allow, it can be implemented within the user device itself as one of the supported sound codecs.

Audio Enhancement Module Interfaces and Performance

Interaction of audio enhancement component 20 with first network 11 and second network 13 is now described in more detail. FIG. 4 shows audio enhancement component 20 deployed in relation to first/second networks 11, 13 which provide a SIP/VoIP environment such as IP PBX, IMS, CAMEL/IN or other SIP environment.

Audio enhancement component 20 interfaces with the networks 11, 13 by way of interface ‘A’ at media gateway controller 21A, interface ‘M’ at media gateway 21B, and interface ‘B’ at configuration management module 23.

Interface ‘A’ comprises signalling to/from the core network 11, 13. Unique identifiers are provided for the first user 10 and second user 14 of a call as well as routing information for RTP packets 17 of the call. RTP packets 17 of interface ‘M’ comprise sound carrying packets to be processed by sound processing engine 22 via media gateway 21B. Interface ‘B’ comprises operation and maintenance connectivity between configuration management module 23 and a network operator's operational support system (OSS) 26.

As previously discussed, audio enhancement component 20 comprises media gateway controller 21A, media gateway 21B, sound processing engine 22 and configuration management module 23.

Media gateway controller 21A comprises interface ‘A’, interface ‘C’ and interface ‘E’. Interface ‘C’ is an interface internal to audio enhancement component 20 between the media gateway controller 21A and the media gateway 21B and comprises a media portion and a control portion. In an embodiment, interface ‘C’ may comprise a physical layer of 1Gb Ethernet with an application layer of RTP over user datagram protocol (UDP) for the media portion and media gateway control protocol (MGCP) over UDP for the control portion. Interface ‘E’ may be used to monitor and control media gateway controller 21A by way of the configuration management module 23.

The media gateway 21B allows the performance of sound processing in creating an RTP proxy in which real time voice data may be extracted for processing and returned to the same gateway for routing. In short, the media gateway is a SIP router for signaling conversion from the network of interest to SIP 16 and also routing the traffic as RTP 17 towards sound processing engine 22.

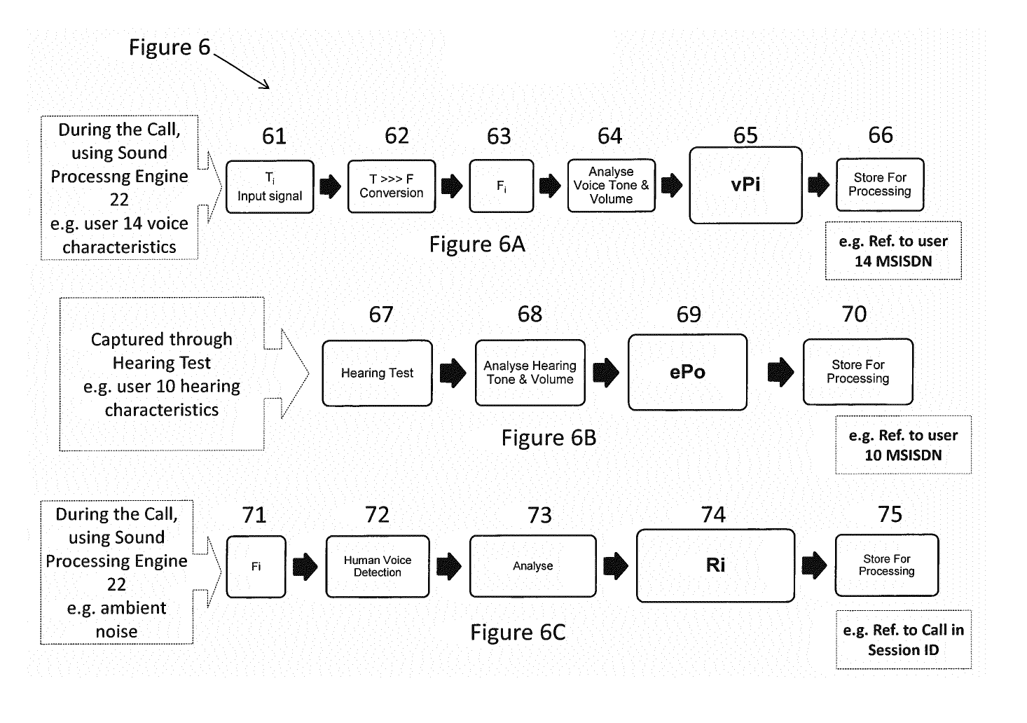

Configuration management module 23 comprises database 25, interface ‘B’, interface ‘D’ and a user interface 24, which may comprise a web portal for example on a laptop or handheld device which may be voice activated and/or used in combination with an accessory such as a headset or other hearing and microphone setup, the user interface comprising interfaces ‘F’ and/or ‘G’. User interface 24 provides user access to audio enhancement component 20. Interface ‘F’ of user interface 24 provides user setup for capturing a user hearing and voice profile (biometrics enrolment) by way of initial and on-going calibration as well as parameters for sound processing algorithms (see later in relation to FIG. 6). Interface ‘G’ comprises administration and support functionality. Interfaces ‘F’ and ‘G’ may be part of the same interface. Database 25 comprises user information in relation to biometric data, and hearing and voice profile information for use with sound processing engine 22 as described later. Interface ‘D’ is for passing sound processing parameters as defined in a user hearing and voice profile on the request of the sound processing engine 22.

Turning to FIG. 5, and in relation to a call from first user 10 (a subscriber 15A of the audio enhancement service) by way of, for example, a mobile origination point (MO) to second user 14, for example a mobile termination point (MT), data flow (50) associated with call initiation and audio enhancement by sound processing engine 22 is shown. Core network 11, 13 has no visibility of the internal functionality of audio enhancement component 20, a network merely has to know which user identifier to use for which user, for example, the MSISDN which is unique for each user.

In the example of FIG. 1, the MSISDN numbers associated with both terminating points 10 and 14 are associated with a session ID for the call by the application server (media gateway controller 21A) and associated parameters are passed to the audio sound processing engine 22 via interface ‘X’. For example, a unique identifier for the first user 10 is provided via interface ‘A’ to media gateway controller 21A and in turn to media gateway 21B via interface ‘C’ and onto sound processing engine 20 via interface ‘X’.

Sound processing engine then requests corresponding biometrics over interface ‘D’ in the form of a hearing and voice profile from database 25 of configuration management module 23 for that user at the start of a particular telephone call. Once the profile is returned to the sound processing engine 20, audio enhancement of RTP packets 17 can proceed in real time.

In the example of FIG. 5, first user 10 therefore benefits from enhanced audio.

For the call to proceed with audio enhancement, database 25 is interrogated for biometrics associated with the both the MO and MT MSISDN numbers.

In an embodiment where both MO and MT are enrolled for audio enhancement, the sound processing engine will apply parameters from the biometric profiles of each user contained within database 25 to both sides of the conversation. This may include employing audio enhancement in relation to a hearing profile, voice profile or both, independently for each user.

Even if a particular user is not registered for voice enhancement, their voice biometric profile may be captured and stored in database 25 against their unique MSISDN number such that whenever they communicate with a registered user, that registered user can benefit from a higher degree of enhancement by the initial input signal conditioning for the unregistered user being optimised for the registered user.

As described, sound processing engine 20 requires a hearing and voice profile in order to be provided with parameters to feed into a sound processing algorithm. Database 25 holds the values associated with each hearing and voice profile of each individual user, for example, by way of a look-up table.

Each user's hearing and voice profile is configurable to their specific hearing impairment both by way of enhancing the voice originating from the user, and the voice delivered to the user. Phone feedback (transducer effect) and/or ambient noise may as an option be taken into account.

FIG. 6 illustrates the processes involved in acquiring a user's hearing and voice profile by way of input conditioning for voice (FIG. 6A), output conditioning for hearing (FIG. 6B) and optional ambient conditioning (FIG. 6C). Any or all of the input, output and ambient conditioning can be enabled or disabled as required by the user. For example, if a user of enhanced audio is holding a telephone conversation and then passes their phone to a friend to continue a conversation, the friend may not require audio enhancement as they may not have impaired hearing.

With reference to FIG. 6A (conditioning the incoming voice through sound processing engine 22 towards user 10 as a registered subscriber 15A with hearing loss), upon commencement and during the call in session, the incoming voice is sampled at step 61 from a user's communications device (14 in FIG. 1), or from another input device associated with user 14's unique identifier, for example an MSISDN number. The signal is converted from the time domain to the frequency domain at step 62 to provide a frequency domain signal, Fᵢ at step 63. At step 64, voice type (for example soprano, mezzo-soprano, contralto, counter tenor, tenor, baritone or bass) and volume is analysed to result in a voice profile at step 65 where the voice profile of the speaker's voice (characterisation of the actuator) is derived. This allows the optional automatic moving of the sound of the originator of the voice (user 14) by one or more frequency (tone) steps as an error function towards the hearing profile of the hearing characteristic of the user receiving or hearing the incoming voice (user 10 in this instance). This voice profile is stored in database 25 with an associated voice originator user id unique to the user in question at step 66. This results in the voice profile not necessarily needing to be derived again if the same user (14) uses the same line (MSISDN) in a future call. Statistical variation of the voice may also be captured. This could indicate that a particular line (MSISDN) is used by multiple people and therefore, for such a line, voice characterisation may need to be performed every time a new call is made as it is not sufficiently predictable which user (voice) will be making the call.

With reference to FIG. 6B (conditioning the signal a user will hear from the sound processing engine 22), an audio hearing test signal is provided at step 67 to a user's communications device, or to another output device associated with user interface 24 of configuration management module 25. At step 68, the hearing tone and volume is analysed to result in a hearing profile at step 69 (characterisation of the sensor—the user's ear). The hearing profile comprises parameters for balancing different frequencies on the sound wave that is presented to a subscribing user. It is a pseudo prescription of the hearing of the user. Any particular user will hear an incoming sound most efficiently and with most clarity if the incoming voice is matched to their hearing profile.

This hearing profile is stored in database 25 with an associated user id unique to the user in question at step 70. The profile may be considered a combination of the user's hearing loss in association with and taking into account the measured transducer and system noise impact involved in the test to give a combined hearing threshold specific to that user at that time tailored to the telecoms network. The combined hearing threshold may be unique to that user. It may be a digital ‘voiceprint’ threshold that is bespoke to the user. The term “threshold” may be considered a hearing threshold, in as much as a level (e.g. volume and/or frequency) at which a user can satisfactorily hear an audio signal, This threshold may be below that threshold of hearing loss. This representation of hearing threshold contrasts with traditional measures such as audiogram given the difference in how the hearing loss is transcribed to work on, be modified and transferred over a communications network.

Further details as to the hearing test performed at step 67 are as follows:

Based on perceived hearing loss of the user (none, mild, moderate, severe or severe-to-profound according to various institutional measures), an initial volume for the hearing test is determined. The initial value may be determined by the user, in some embodiments. In some embodiments, the gender and/or age of the user may be alternatively or additionally taken into account when setting the initial volume.

The hearing test commences:

### 1. Start Hearing Test

a) Instructions to the user for the hearing test may be provided via user interface 24.
b) The media gateway controller 21B places a call to the user's phone.

As would be understood, it is the underlying network for example a broadband network that provides the user interface 24 (e.g. web portal to a user or voice activated interface), and a voice communications network for example telephony or VoIP that provides voice to a user handset or device. These networks run on different clocks e.g. a browser or laptop clock versus a telecommunications network clock. Therefore, knowledge of the delay between a user hearing a tone on their device, and acknowledging the tone being heard on the web portal may cause errors or inaccuracies in the hearing test where time to react to an automated test, which could be altered by differing clock values between networks, can determine an erroneous true or false outcome at a particular hearing test frequency which may affect measured threshold levels of a user's hearing capability and hence adversely affect that user's biometric profile (see later). Therefore, master clock and timers for the client and server (media gateway controller) platforms are synchronised.

One way to synchronise clocks across a server and user device is as follows. The user (client) device, at the time of requesting commencement of a hearing test, requests a plurality of pings from the server (for example five). One or more of the plurality of pings may comprise a spread of frequencies representing voice or white noise. This may contrast with standard hearing tests which uses specific single frequency tones. The server sends a ping packet with a data payload of the current server time. The ping packet is received by the client device and sent after a set time gap (for example one second). After a further set time gap (for example two seconds) a replica of the ping packet is sent back. This can be repeated several times such that the server receives a plurality of ping packets, each relative to the corresponding originating packet sent back form the client device. From these packets, the server can calculate the transmission travel time from user to server as well as the drift in the clocks at the client and the server. This helps avoid the previously mentioned erroneous true or false test results.

Further, as volume of a test decreases (see below), the time delay in a keypress for a missed hearing test is important for a test outcome. Test results are fine tuned with half steps (5 dB as opposed to 10 dB). The time taken to test can be reduced by having accurate clock syncing information so that the number of half steps can be reduced.
c) Deactivate the Sound Enhancement function towards the user's phone
d) Stream reference speech to the user's phone and request user to adjust the sound volume in the handset for comfort in hearing the reference speech
e) Synchronise the timers & test for hearing threshold @ 500 Hz
f) Synchronise the timers & test for hearing threshold @ 1000 Hz
g) Synchronise the timers & test for hearing threshold @ 2000 Hz
h) Synchronise the timers & test for hearing threshold @ 3000 Hz
i) Synchronise the timers & test for hearing threshold @ 6000 Hz
j) Activate the Sound Enhancement function towards the user's phone
k) Synchronise the timers & stream reference speech to the user's phone and via user interface to request the user to adjust the volume index

### 2. Hearing Test is Complete

On completion of the above hearing test, parameters are captured as a hearing profile (biometric data) within database 25 of the configuration and management module 23. The parameters may be dependent on one or more of user hearing loss, system noise and transducer effects.

Typically, for the hearing test, the stimuli will be ⅓ octave wide bands of noise centred at 500, 1000, 2000, 3000 and 6000 Hz or higher. Preferably, the duration of each test is about 1000 ms, including 20 ms ramps for increasing and decreasing volume of stimuli between background noise and −60 dB as an example. The spectral slopes of the stimuli are preferably steep, preferably 90 dB/oct or more.

The ⅓ octave wide noise is, in effect, white noise comprising a mix of one or more human voices and is tested at frequency bands up to the capability of the communication system being used. White noise comprising human voices provides the benefit of a more real world test that reflects how a conversation is delivered to the user and enables a more accurate characterisation of both actuator parameters (vocal chords) and sensor parameters (user ear). The white noise used for each test may characterise alternative sounding pronunciation (differing alphabets) sent to user for fine tuning of hearing profile parameters.

The suggested order of testing is: 500, 1000, 2000, 3000, 6000 Hz or higher for a wideband or super-wideband voice codec or up to 3000-3400 Hz for a narrowband codec. Narrowband and wideband codes being the typical codecs used in legacy telecoms systems. A test can be tailored for the underlying communication means such as the network capability for transporting audio be it via a narrower or wider band. Measurements at one centre frequency are preferably completed before the next centre frequency is selected.

More detailed procedure for each test frequency is given below as an example implementation:
a) The sound is presented at the initial level estimated as above
b) If a response of “yes” is given within, for example, 2 seconds of the end of the sound, this is taken as a “hit” and the level of the next sound is reduced by 10 dB. If there is no response within 2 seconds after the end of the sound, this is scored as a “miss” and the level of the next sound is increased by 10 dB.
c) The next test sound may be presented after a variable time interval, to avoid the user responding “yes” at an anticipated time. If the response to a previous sound is a hit, the next sound is presented after a delay preferably randomly selected from the range 0.5 to 2 seconds after the “yes” response. If the response to a previous sound is a miss, the next sound should be presented after a delay preferably randomly selected from the range, for example, 2.5 to 4 seconds after the end of the previous sound.
d) Step (b) is repeated until at least one hit has occurred, followed by a miss. After the miss, the signal is presented with the level increased by 10 dB.
   a. If the response is a hit, the signal level is decreased in 5 dB steps until a miss occurs. The lowest level at which a hit occurs is taken as the threshold level for that frequency.
   b. If the response is a miss, the level is increased in 5 dB steps until a hit occurs, and then the level is decreased in 5 dB steps until a miss occurs. The lowest level at which a hit occurs is taken as the threshold level for that frequency.

This procedure is repeated for each test frequency in turn. However, if the initial response to the previous test sound is a miss (meaning that the starting level was too low), the starting level for the current centre frequency is set to the threshold level at the previous frequency plus a predetermined amount, for example plus 25 dB.

The hearing test may be repeated at a later time which allows the user to see the long term change in their biometrics parameters and reduces the standard deviation in the captured threshold parameters.

The final result of the combined hearing threshold or ‘digital voiceprint’ may then be visually and/or otherwise presented as specific to that user. The result can be interpreted including, for example, listening to the test result, saving the test result, cancelling the test result or redoing the test. The hearing test results can then be listened to to compare the processed versus the unprocessed voice. This may or may not lead to the recorded hearing threshold also being fine-tuned further, for example using adaptation of compression ratios and/or frequency levels such that the digital voiceprint or the original combined hearing threshold more accurately reflects user preferences and tonality which can and may be adapted over time as hearing loss or needs change. This digital fine tuning is possible once the combined hearing threshold reflecting personal hearing loss or needs alongside system noise and transducer effect has been measured as above. In other words, a user may interface with a screen to record and map their hearing loss. That is the combination of system “noise” plus transducer impact is used to create a digital threshold. The visual output may be considered a “graphic” representation of the conjoined hearing threshold of hearing loss and device transducer effect.

With reference to FIG. 6C (taking into account at least one of ambient noise, signal to noise ratio, echo, packet loss and other detrimental effects), at step 71, a frequency domain signal, F₁ which may be the same signal as that of step 63, or may be a newly acquired signal to cater for live conditions, is processed by a standard human voice detection algorithm at step 72, and analysed at step 73 to result in an ambient noise profile at step 74 (characterising the channel used for audio delivery). This noise profile is stored in database 25 with an associated user id unique to the user in question at step 75. As an extension to ambient noise conditioning, an optional alarm or other signal indicative of an audio signal to noise ratio that makes cognitive information exchange difficult may trigger certain recorded messages to be sent to the users on a call so that they are aware of the ambient noise issue and they can move to an environment where noise is less perceptible. The user may accept or reject the alarm and hence provide feedback such that future alarms occur at an appropriate time when the individual user would have find cognitive information exchange difficult. Other functionality such as the ability to record a conversation may be provided to aid a hearing impaired user to review and verify the conversation after the event. For example, calls can be recorded and stored and in combination with feedback from the user, knowledge derived to pre-define and anticipate future situations in which a particular voice experience occurred could and therefore could be overcome—in effect the sound processing engine 22 can learn how to recognise, avoid or compensate for such potentially difficult voice scenarios by way of artificial intelligence. Over time this knowledge databank can be built up and stored in database 25, shared and used to develop and enhance the audio enhancement and processing algorithms for more generic use in other situations—such as fine tuning a hearing threshold for a range of voice ambient situations that cater for the environment and/or the network signal strength at that time, whether over a fixed, mobile or wireless network for example. Typically, the use of AI to improve user experience is not used real-time in the telecoms/IP network, therefore the present disclosure can improve the voice experience for those with addressable hearing loss needs.

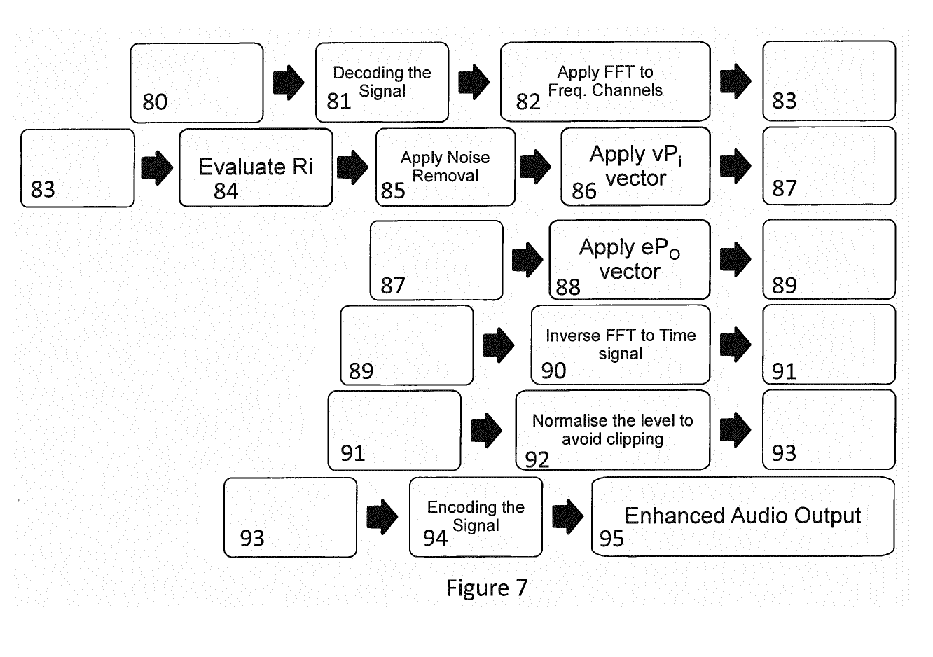

FIG. 7 illustrates processing steps undertaken by sound processing engine 22 when it is enhancing audio. As will be shown, parameters derived in the profiling process of FIGS. 6A, 6B and optional 6C are used to enhance audio to the needs of the receiving user (user 10 in the example of FIG. 1).

At a first step, 80, an input audio signal from a user (14) to be sent to a subscribing user (1.0) is acquired, and decoded at step 81. The audio signal is transformed into the frequency domain at step 82 to result in a frequency domain signal at step 83. At step 84, ambient noise is evaluated in the same manner as FIG. 6C, and the noise is removed at step 85. Thereafter, voice profile parameters as stored in database 25 during step 66 of voice conditioning are applied (step 86) to produce an enhanced voice output at step 87 (still in the frequency domain).

At step 88, hearing profile parameters as stored in database 25 for the recipient (subscribing user 10) during step 70 are applied to the enhanced voice output, and at step 89 an enhanced voice output is provided (in the frequency domain). At step 90, the enhanced voice output is transformed into the time domain so that an enhanced time domain signal results at step 91. At step 92, the enhanced voice output is normalised to avoid clipping so that a normalised voice output is provided at step 93. Finally, the output is encoded for the underlying transmission protocol at step 94 and enhanced audio (termed a voiceprint) tailored for the hearing of the subscribing user recipient (10) is provided at step 95.

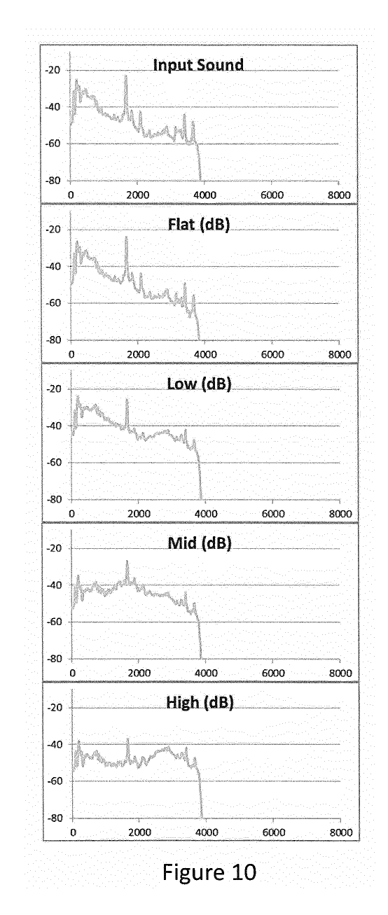

By way of examples, FIGS. 9 and 10 illustrate the waveforms produced by the sound processing engine (frequency domain) when providing enhanced audio.

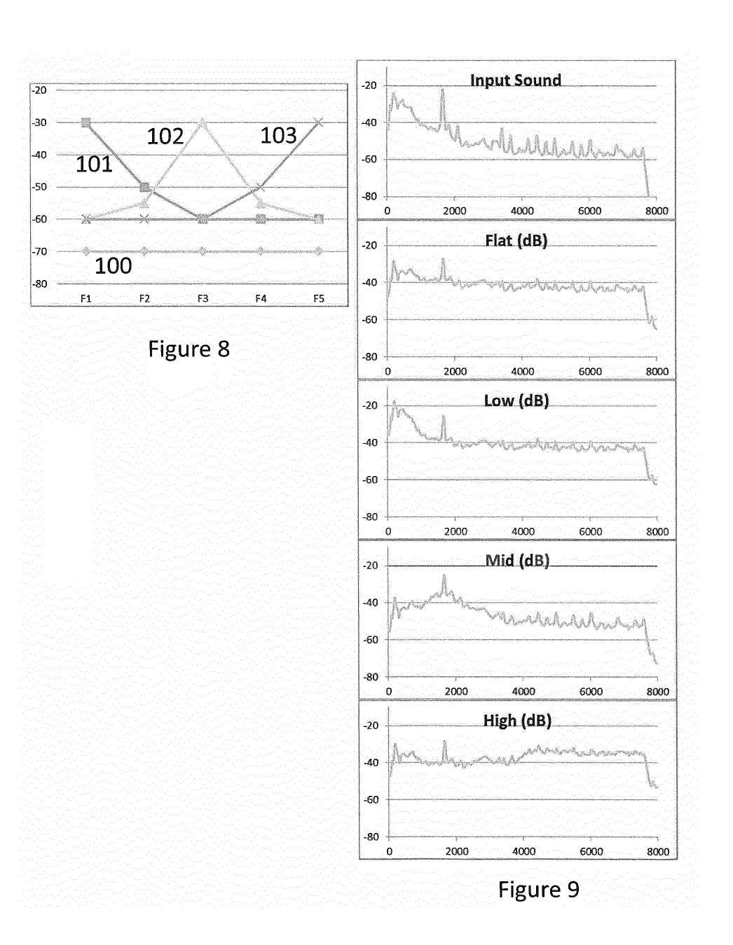

Firstly, turning to FIG. 8, frequency response of the audio enhancement may be tailored by any or all of the response curves shown. Frequency bands are represented in the horizontal axis, and the vertical axis show the thresholds (the limit of hearing of a user for that frequency) as determined during a hearing test as previously described. The scale on the threshold axis represents a sound pressure level indicative of the sound volume.

A “flat” response (no variation in the frequencies) is shown by 100. “Low” is enhancing the sounds at lower frequencies (101), “Mid” enhances the mid bands (102) and “High” enhances the higher bands (103).

FIG. 9 illustrates the frequency spectrum of sample real time sound passing through sound simulator processing using wideband voice processing at 16 kHz. FIG. 10 illustrates the same using narrowband voice at 8 kHz. The narrowband and wideband frequencies shown are for illustrative purposes only. Many other bandwidths of input signal may be dealt with.

When undergoing real time enhancement of audio signals such as speech or music, any or all of the flat, low, mid and high filters can be applied at any time depending on hearing and voice profile parameters stored in database 25 for a particular user.

As well as the derivation of the voice profile and hearing profile for a particular user as described above, an input voice to be sent to a subscribing user, may optionally, in real time, have its input tone moved towards the voice type of the recipient of the audio as previously described in relation to steps 64 and 65. This is by way of an error function acting on the audio signal and applied in sound processing engine 22, for example across filter banks. The variation in tone desired can be stored alongside the user's other profile data for future use. The tone variation may be carried out automatically when a subscribing or non-subscribing user calls a subscribing user from a known MSISDN. The voice type from a particular MSISDN can be stored in database 25 such that if a different user calls from the same MSISDN, the automatic tone variation can be turned off by way of artificial intelligence built into sound processing engine 22. An example implementation may be to observe the standard deviation of the parameters representing the voice profile and compare this with a learnt threshold. Where the standard deviation value exceeds the learnt threshold, sound processing engine 22 can automatically turn off tone variation as it will assume a different person is likely to be using this incoming line.

As well as a hearing profile and ambient profile in relation to an input to be sent to a subscribing user, the volume of voice to be received can be adjusted a number of ways:
Simply amplify the volume of the output at the last processing stage (step 92)
Amplify the digital range of the input signal after removal of ambient noise (step 85). The amplification may be based on an error function using a feedback parameter evaluated over a time period, for example, 20 processing time intervals in the current conversation.
The above feedback parameter may be stored in the user's profile information in database 25 as a long term variable.
Over a longer period of time, for example many conversations, the initial parameters as used by sound processing engine 20 can be tailored based on real world experience of conversations between certain users, providing an optimised voiceprint for a user.
Further, parameters of a hearing profile can be altered over time to account for degradation in a user's hearing whether or not the user undertakes a subsequent hearing test to update their hearing profile. For example, a user's hearing threshold worsens with age. The disclosed method and system can measure threshold loss over time, and, via the combination of user feedback, interrogation and artificial intelligence, hearing loss data in relation to that user's use of the phone, their age, sex and frequency loss is used to create a predictive, dynamic hearing threshold that can automatically adapt to that user's age and sex by virtue not just of its predictive abilities but by comparing such data to the relevant peer group. In essence, the algorithms link in with the AI by allowing interpretation not just of the user's hearing characteristics but also of the network signalling strength for a particular conversation (e.g. packet loss in fixed network or RF signal strength in wireless networks) such that it can predict that if the signal is poor, the hearing threshold can be shifted to a lower level to enhance the audio processing to deliver a more pronounced (higher volume) voice signal. This measure of hearing threshold, its adaptation of such a threshold over time (age of user) and against signal strength is unique since it allows the adjustment of user hearing profiles both over time to cater for degradation in user hearing, and for the immediate conversation to hand.

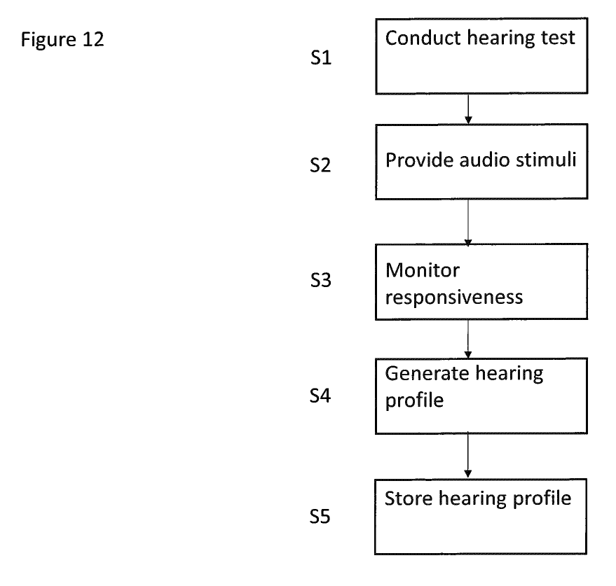

A hearing test, and use of results of the hearing test in order to modify audio signals to a user will now be described in more detail with respect to FIG. 12. It will be understood that the methods now described can be used in conjunction with the method described, for example, with respect to FIGS. 6A to 6C and FIG. 7 (and indeed any other embodiments of the description).

The method described with respect to FIG. 12 relates to a hearing test carried out between a network entity, for example a server residing in a communication network, and a user communicating with the server via a user device. The communication network may be a telecommunication network. The user device may be a phone, such as a mobile phone; alternatively the user device could be a laptop, tablet etc. It will be understood that by carrying out the hearing test over the network, and with a user's device, then this gives a more accurate portrayal of how the user's hearing is affected in real-world conditions. It also takes into account aspects specific to a particular user. For example, the hearing test may take into account network effects such as interference or noise, or aspects particular to a user's particular network provider such as particular compression algorithms they use. It may also take in to account aspects related to a user's specific device, for example transducer effect of the device's speakers. It may also take into account aspects of the user's other hearing devices, such as hearing aids and/or implants.

As shown at S1, a hearing test is conducted for a user over a communication link established between a network entity (for example an entity or server comprised in audio enhancement component 20) in a communications network and a user device of a user (e.g. user 14). (The communication link may be established between the network entity and the user device by the user initiating contact with the server, for example by the user phoning a contact number of a service provider of the hearing test. Alternatively, the service provider may call the user on their user device, for example at a pre-arranged time. However, the link is established, it will be understood that the hearing test is conducted over a link that is established between a network entity in a communications network and in combination with a user device of a user.

In some embodiments, the hearing test may use a platform. This may be the same media enhancement platform as is used during calls or similar to such a platform. The hearing test may alternatively or additionally use a web based testing portal. This may initiate and/or receive automated calls to and/or from the user's phone. This portal may guide the user through the test process via one or more on-screen prompts or instructions. This portal may do this by interacting with the media enhancement platform.

The hearing test may be carried out in an automated or semi-automated fashion. For example, the user may follow automated prompts from the server/service provider. Alternatively, the user may speak directly with a human operator of the service provider who conducts the hearing test. The prompts may be visual prompts and/or spoken prompts. The prompts may be displayed on a user device of the user. The prompts may be provided on the same user device which is in communication with the server for conducting the hearing test. Alternatively, the prompts may be provided on a separate user device. For example, the user may follow prompts displayed on a laptop or tablet, in conjunction with carrying out the hearing test via their user device which has the communication link with the server of the service provider.

As shown at S2, the hearing test comprises providing audio stimuli to the user. The audio stimuli are provided to the user device at a plurality of test frequencies.

According to some embodiments the audio stimuli comprises white noise. The white noise may be based on one or more human voices, which more accurately mimics the type of sounds that a user will typically hear on their user device, such as during a telephone call. According to some embodiments the audio stimuli comprises ⅓ octave wide bands of noise.

According to some embodiments the providing audio stimuli to the user at a plurality of test frequencies comprises providing audio stimuli at two or more of 500 Hz; 1000 Hz; 2000 Hz; 3000 Hz; 6000 Hz. These values are by way of example only and different values may be used, including frequencies lower than 500 Hz and higher than 6000 Hz. For example, values higher than 6000 Hz may be used for a wideband or super-wideband voice codec, or up to 3000-3400 Hz for a narrowband codec. The white noise may be played at the test frequencies in a pre-defined order e.g. 500 Hz; 1000 Hz; 2000 Hz; 3000 Hz; 6000 Hz. The change of frequency may be conducted in a step-wise fashion.

At S3, responsiveness to the audio stimuli received from the user device is monitored. This may also comprise measuring responsiveness. The monitoring responsiveness effectively checks whether the user has heard the audio stimuli that has been played to them. The monitoring may for example include monitoring for feedback from the user, such as a key-press on their user device (which may be the user's phone or associated laptop, tablet etc.) or for a speech response from the user.

Prior to playing audio stimuli to the user, information may be obtained from the user regarding their hearing ability. In some embodiments, this may be at least in part, assumed and/or pre-defined also by gender and/or age. This may include obtaining an indication of hearing loss of the user. This may include obtaining information such as whether the user's hearing loss is none, mild, moderate, severe or severe-to-profound according to various institutional measures. The user may be requested to provide this information. The indication of the user's hearing loss can be used to determine an initial volume of the hearing test. The volume of the audio stimuli can then be adjusted during the hearing test, in response to the monitoring of responsiveness. For example, in response to a positive response from the user the volume may be decreased for the next stimuli. This may occur in 5 dB steps. Of course, the step change may be by other amounts in different embodiments. In response to a null response from the user, the method may comprise increasing the volume of the audio stimuli. The increasing the volume may comprise increasing the volume in 10 dB steps. Of course, the step change may be by other amounts in different embodiments. In some embodiments the adjustment of volume of audio stimuli may occur at each test frequency.

According to some embodiments the duration of each audio stimuli is 1000 ms or about 1000 ms. Of course, this is by way of non-restrictive example and in other embodiments the duration of the audio stimuli could take other values. There may be a change or a variation of volume within each audio stimuli. For example, each audio stimuli may include one or more ramps of increasing/decreasing volume between a background noise level and 60 dB (or about 60 dB). Again, this value of 60 dB is by way of example only and in other embodiments different values may be used.

Based on the hearing test, and as shown at S4, a hearing profile may be generated for the user. This may be considered a hearing profile threshold. The hearing profile comprises an accurate measure of the user's hearing loss, taking in to account network effects such as signal quality, network noise etc. as well as effects pertaining to the user's device e.g. transducer effect.

Once the hearing profile is generated it can be stored in a memory of the network entity. This may be the same network entity which had the communication link with the user device of the user and which conducted the hearing test. Alternatively, it may be a different network entity or on a device. This is shown at S5. The hearing profile may additionally be stored at other entities, including other network entities or at the user device. In storing the hearing profile an association may be made between the user and/or user device. For example, the association may be stored in a look-up table. This enables that user's hearing profile to be obtained and used when transmitting and modifying audio signals to the user device of that user. In other words, the stored hearing profile is available for modifying of audio signals to the user device. Of course, the network entity may store a plurality (which may be hundreds, thousands, millions etc.) of such associations between users and/or user devices and associated hearing profiles. According to some embodiments the information associated with the user comprises an identifier of the user. The identifier may be a unique identifier. The identifier may be for example a name of the user. The identifier may additionally or alternatively comprise an identifier of the user device of the user. For example, the identifier may comprise an MSISDN of the user device.

In some embodiments, the hearing test may comprise processing and fine tuning of the output of the hearing test. This may take place whilst the network entity is in communication with the user, or could take place after the user has completed listening to the audio stimuli. This may enable fine tuning of the hearing profile to the user's natural ear, and/or to fine tune the hearing profile to a further hearing device of the user (e.g. hearing aid or cochlear implant). In this regard the method may comprise visually displaying results of the hearing test to the user and/or an operator in communication with the network entity. The fine tuning may be carried out by the user, for example via their user device or a separate laptop, tablet etc. Additionally, or alternatively the line tuning may be carried out by an operator who is in communication with the network. For example, the operator may be an employee of the service provider of the audio modification service.

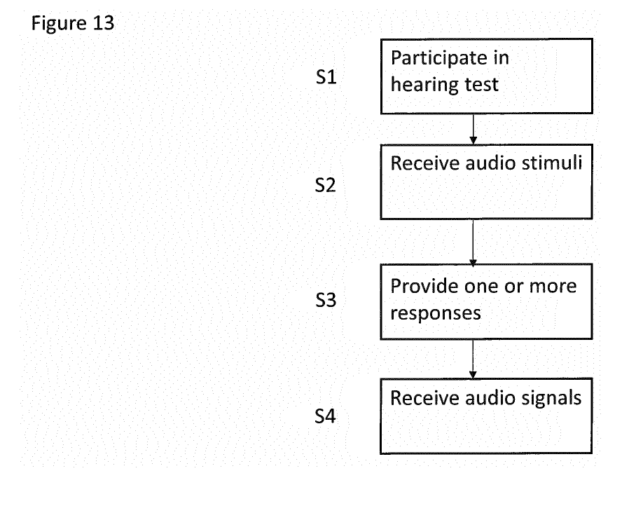

FIG. 13 is a flow chart showing a method according to an example, viewed from the perspective of a user device.

At S1 a user, via their user device, participates in a hearing test on a communication link established with a network entity.

At S2, the device receives audio stimuli at a plurality of test frequencies over the communication link. That is the hearing test is carried out in a manner as described in detail above.

At S3, the user provides one or more responses to the audio stimuli to the network entity. The responses may be provided via the user device on which the user is listening to the audio stimuli, or the responses may be provided via a separate device of the user e.g. a laptop or tablet of the user.

Subsequently the user can receive, at their user device, modified audio signals as shown at step S4. These modified audio signals are modified based on the hearing profile that is created for the user following the hearing test, as described in detail above.

The modified audio signals can be delivered to the user device of the user in real time (and ultimately to the user's natural ear, hearing aid or implant etc.). Say for example a user who has carried out a hearing test and has a stored hearing profile is user A. User A's identifier (e.g. MSISDN) is stored in association with the hearing profile of User A in the network. When a second user, User B, calls User A then, User A's hearing profile is retrieved from memory and the call can continue with User B's voice (and indeed any other audio signals), being modified in accordance with User A's hearing profile (or “voiceprint”). The modifying of an audio signal may comprise any one or more of: filtering the audio signal; adjusting the amplitude of the audio signal; adjusting the frequency of the audio signal; adjusting the pitch and/or tone of the audio signal. According to some embodiments the audio signal modification may be carried out by a sound processing engine in the or a network entity.

According to some embodiments, ambient noise at a location of the user device may be recorded. The ambient noise may be recorded using one or more microphones of the user device. The ambient noise information can be transmitted to the network where it may be stored. The ambient noise information may be collected and stored in real time during a phone call, for example. The ambient noise information can then also be used in delivering modified audio signals in real time to the user device.

Some further details of audio signal modification will now be described, by way of example.

### Overview of an FFT-Based Signal Processing Function

Digital audio is usually regarded as consisting of a time series of audio samples. In order to preserve the illusion of a continuous sound, a new sample has to be converted to analogue every time period, this period being the reciprocal of the sampling frequency. However, the actual processing of the audio in this algorithm is not necessarily on a continuous sample-by-sample basis, but by “frames” of audio samples, which are 128 samples in length. Each frame, both reading and writing may be overlapped with the previous frame by 50%. So each sample in the audio stream may actually be sent for processing twice.

The processing rate of the frames may be much slower than the audio sample rate:

FsFFT=Fs/(framelength/2)

where FsFFT is the sampling rate of the frame, Fs is the sampling rate in Hz (of the audio samples) and framelength is the number of samples in the frame. The sampling rate of the processing may always be one value, for example 16 kHz, but that if the audio stream arrives at any other rate a sample rate conversion may be required between the two rates.

In embodiments, an FFT (Fast Fourier Transform) length of 128 samples at 16 kHz may be used. However, due to the context in which this algorithm is required, it may be necessary to adapt the number of audio samples which are inserted into each FFT frame.

With the two different sample rates running simultaneously, there may need to be two processes running in parallel to keep the processing continuous.

(1) An interrupt-driven process that takes the sample from the input stream and puts it in an input buffer, along with taking a sample from an output buffer to place in the output stream.
(2) Frame based processing, which may be accomplished before the current input/output sample buffers overfill or empty, respectively.

The minimum audio time delay between input and output of this form of “overlap-add” processing is, in an example, 1.5 times the frame length. The buffer pointers for the interrupt-driven process may be updated within one sample period (1/Fs) once the full/empty flag occurs, otherwise stuttering of the audio may occur. If the frame processing is sufficiently powerful, the frame may be processed before the input/output buffers have run out or filled up.

In the following pseudocode example of the processing, the major function of a step is indicated by a Roman numeral in bold (0, I, II, III, IV, V, VI) and each sub-step of the processing is numbered in normal type, eg (1). If there is conditional processing in a step, the conditions are indicated by numbers after the decimal point eg (1.1, 1.2, . . . ).

(0) Start: assuming that there has been accumulated either:
(0.0) 32 samples of audio at a sampling rate of 8 kHz or
(0.1) 64 samples of audio at a sampling rate of 16 kHz
in a buffer called input(i), i=0 . . . 31 or 0 . . . 63, depending on sample rate.
Then the process continues as follows
(I) All audio samples need to be converted into a linear representation in single precision (4-byte) floating point format samples, hence any instantaneous compression needs to be undone.
(1.1) if samples arrive in “mu-law” or
(1.2) “A-law” coding,
(1.3) any other non-linear coding format
These can be undone with the inverse function (using a look-up table).
Pseudocode: xt_lin=inv_law(input);
where xt_lin are the sample values in linear format, and input is the incoming latest buffer. inv_law( ) is the mapping function between compressed sample value (8 bit integer, hence 256-entry table sufficient) and the floating point representation of the linear sample value.
In embodiments, this is done one buffer at a time, to prevent repeated function calls for every sample.
(II) Data is expected to arrive at one of two sampling rates, either 8 kHz, (standard telephone rate) or 16 kHz (wide bandwidth). Hence, in embodiments, all processing is performed at a 16 kHz sampling rate in fixed length “frames”.
(1) Sample-rate conversion may be performed within the FFT structure.
Each FFT frame is half filled with the most recent input buffer, the remaining half is filled from the previous input buffer. Thus, there may be a 50% overlap of samples between adjacent frames (each input buffer appears in two consecutive frames). There may also be “zero-padding” outside of the inserted audio samples.
(2) Construct an empty frame of length 128 samples once to hold the linear-coded audio samples. (index 0 to 127)
Pseudocode: x=zeros(128,1);
(3.1) If the audio is at 8 kHz sampling rate, after the arrival of the latest 32 audio samples, then these samples can be inserted into input(0 . . . 31) at index positions 65, 67, 69 . . . 127 in x. For the very first frame in a new processing sequence, the rest of the array may be left unfilled (filled with zero's). For all other frames, index positions 1, 3, 5 . . . 63 may be filled with the 32 samples from the previous input buffer (0 . . . 31).
(3.2) If the audio is at 16 kHz sampling rate, the latest 64 audio samples may be inserted into input(0 . . . 63) and place them at index positions 64, 65, 66, . . . 127 in the frame. For the first frame in a new processing sequence, the rest of the frame may be left unfilled (0 . . . 63). For all other frames, index positions 0, 1, 2, 3, . . . 63 may be filled with the 64 samples of the previous input buffer.
(4) Generate a “window” function. This may be a symmetric ramp in shape and a 0-pi representation of a sine wave. This can be pre-calculated into a small array, and may be used again in the processing.
The sample values of this window at index i are called W(i).
Pseudocode: for i=0, 1, 2 . . . 127

W(i)=sin((i+0.5)*(pi/N))

where pi=3.14159265 and N is the audio array size (N=128).
(5) The frame array is “windowed”. This is a sample by sample multiplication between the audio stream and the window W(i).
Pseudocode: xw(i)=W(i)*x(i); for i=0 . . . 127
(III) Perform a forward FFT on this data frame.
(6) Pseudocode: xf=fwd_fft(xw);
The FFT function will generate a same-length array, but the data type will change to comprising complex numbers.
(a) The output array is considered as two halves, positive frequency and negative frequency. For each point in the output array, its equivalent frequency can be calculated as:

f(i)=i*Fs/N for i=0,1, . . . 63 (2)

f(i)=(1284)*Fs/N for i=64,65, . . . 127 (3)

where Fs is the sampling rate (16 kHz), and i is the index into the 128-point array (assuming that the function has returned the full array. N is the array size (N=128). Equation (2) defines the “positive frequency” side of the FFT array while equation (3) defines the “negative frequency” side of the array. f(i=0) is 0 Hz, and therefore a real number, representing the average level (DC level).
Using Fs=16,000 and N=128, then the “bin spacing” or (f(i+1)−f(i))=125 Hz.
(b) Some libraries may include an FFT function explicitly designed for audio, more specifically for real-only data. They will produce a half-sized array comprising just the values for the positive frequency. Internally, such library functions will perform the necessary manipulations on the negative frequency components to produce the correct forward and inverse transforms, thereby saving processing power.
(c) If the returned array from the FFT has both positive and negative frequency components, any calculation performed on a frequency point in the positive frequency domain does not have to be repeated in the negative frequency domain, just the complex conjugate of the equivalent positive frequency point needs to be copied across.
(6.1) If the input audio stream was originally sampled at 8 kHz, then the components in the FFT array where f(i)>4000 (Fs/2) will need to be set to zero (potentially both halves of the array). This is to remove “aliasing”; performing a sample rate conversion from 8 kHz to 16 kHz.
Pseudocode: i_stop_pos=round(4000*Fs/N);
i_stop_neg=round (128−(4000*Fs/N));
xf(i>i_stop_pos & i<63)=0;
xf(i<i_stop_neg & i>63)=0;
   The rounding function is used to ensure that no fractional indices are generated, and guards against future changes in sample rate or N.
   (6.2) If the input audio stream was originally sampled at 16 kHz, then no processing is necessary.
   (IV). The core of the code: software to implement insertion gain and compression during the FFT. (Effectively a loop back function if no processing inserted here)
   The compression system here is designed to operate in the frequency domain, but splitting the audio signal into 4 channels, calculate the short-term channel power and on the basis of this, apply a dynamically varying gain that maps the audio signal back into audibility and comfort for the, for example, hearing-impaired user.
   Software for one-off pre-calculations necessary for each user
   Every user will have different hearing characteristics, thus for every user a unique hearing aid setting may be calculated:
   (A) Insertion gain (IG) for “65” dB SPL speech, IG65, as a function of FFT frequency
   The precise value of gain as a function of frequency is calculated via the audiogram measure.
   Pseudocode: [freq_ig, gain_dB]=IG65(audiogram, age, hearing aid experience);
   Whereby, freq_ig may be on a logarithmic scale, and gain_dB will express the gain in decibels, a logarithmic function of linear gain.
   Pseudocode: gain_dB=20 log 10(gain_linear);
gain_linear=10\hat{ }(0.05*gain_dB);
   This gain may be applied in the frequency domain to the FFT of the audio frame. Therefore, the gain values are interpolated from the [freq_ig, gain_dB] grid to the linear frequency grid of the FFT.
   This is done with two different methods: a first method is to interpolate the linear gain on a linear frequency scale, or a second method of interpolating the logarithmic gain (dB) on a logarithmic frequency scale.

### Given:

f(i)=i*Fs/N for i=0,1, . . . 63 (2)

and

f(i)=(128−i)*Fs/N for i=64,65, . . . 127 (3)

(assuming 2-sided FFT calculation)
then

### Pseudocode:

If (f(i) <min(freq_ig))

Glinf(i) = gain_lin(min(freq_ig));
Glogf(i) = gain_dB(min(freq_ig));

elseif (f(i) > max(freq_ig))

Glinf(i) = gain_lin(max(freq_ig));
Glogf(i) = gain_dB(max(freq_ig));

else

scale
Glinf = lin_interp(freq_ig, gain_lin, f);
Glogf = lin_interp(log10(freq_ig), gain_dB, log10(f));

end

In the first ‘if’ loop it may be determined whether the handle gains are for frequencies below the lowest of the IG65 array. If the condition is met then the logarithmic gain may be interpolated against a logarithmic frequency using minimum frequency values.

The second ‘elseif’ loop will determined whether the handle gains are for frequencies above those of the IG65 array. If the condition is met then the logarithmic gain may be interpolated against a logarithmic frequency using maximum frequency values.

If neither of the conditions are met then the values may be linearly interpolated.

Where values of gain are required at frequencies outside of the original insertion gain array then there is no extrapolation, but the same gain value is extended from the relevant end of the insertion gain array.

Care may be taken that the log 10(f) or log 10(freq_ig) does not get violated if f=0 or f<0, as this could cause errors.

Pseudocode for linear interpolation:

NewY(i)=OldY(f(j))+(OldY(f(j+1)−OldY(f(j)))*(NewX(i)−OldX(j))/(OldX(j+1)−OldX(j));

where OldX(j) and OldXf(j+1) are X points within the known (x,y) function, which bound the value NewX(i), where NewY(i) is desired to be calculated.
(B) Calculate the channel levels for a speech-shaped noise after application of IG65.

This forms part of a calibration procedure. There are two principle stages of gain applied to the FFT array. (i) the prescribed insertion gain (for 65 dB SPL speech) and (ii) the dynamic compression gain. The user-specific insertion gain may be applied before the dynamic range compression software. For a speech input of 65 dB SPL, the combinations of gains need to be the same as the prescribed insertion gain. A correction factor may be calculated so that the dynamic compression gain is 0 dB when the channel power for the compressor is that generated when a 65-dB SPL speech noise is applied. Hence the channel levels are calculated under such a circumstance. Although this can be done in the FFT domain, in preferred embodiments it is completed with a signal file with the same digital RMS as the level at which the insertion gains are specified. MAS can supply a 2-sec noise file with the desired spectrum, but which may be scaled before use, depending on defined reference levels. Channel edge frequencies may be calculated for the compression system. This allows the audio signals to be split into 3 or 4 separate channels in the FFT processing in order to manipulate them semi-independently. Since the calculations are completed in the FFT domain, the bandpass filtering has already been performed, but on a fixed, linear frequency grid. To calculate channel powers, the power from the individual FFT bins that lie within the band-pass section of our desired channels may be summed. Although the power is summed in the FFT bins, the “edge frequencies” of the channels are half-way between “bins” of the FFT, at n*125+125/2 Hz, where n is an integer. (a) POTS, where speech occupies 300-3400 Hz, and transition bands at the edge of the signal are allowed for.

Frequency span FFT bin numbers (called ChanjFFTbin{Start/End})

### Channel (1) 250 to 750 Hz 2-6

Channel (2) 750 to 1500 Hz 7-12 (NB do not double-count bin at 750 Hz)
Channel (3) 1500 to 3500 Hz 13-28 (NB do not double-count bin at 1500 Hz)
Channel (4) 3500-3875 Hz 29-126 (Dummy channel, should not be carrying signal)
(b) wide-bandwidth speech:

Frequency span FFT bin numbers (called ChanjFFTbin{Start/End})

### Channel (1) 0 (DC) to 750 Hz 0-6

### Channel (2) 750 to 1500 Hz 7-12

### Channel (3) 1500 to 3500 Hz 13-28

### Channel (4) 3500 to 7875 Hz 29-126

So process the noise calibration signal in the FFT domain and form the average level of the channel powers.

### Pseudocode:

(i) The array is initialised (only needed at the very start).
for j=1, 2, 3; ChannelPower65(j)=0; end
   (ii) Apply insertion gain to xf:
xf_ig(i)=xf(i)*Glin(i);
   (iii) Calculate the power in each FFT “bin”
BinPower(i)=xf_ig(i).*conj(xf_ig(i);
   (iv) Sum the powers from each bin into its relevant compression channel. Start and end bins are given above, in variables ChankFFTinStart to ChanjFFTbinEnd
for j=1, 2, 3, 4
ChannelPower65(j)=sum(BinPower(i));
   The ‘i’ value will span several bins.
   The vector ‘ChannelPower65’ is calculated for each frame generated in processing the calibration signal (indexed by k).
   Then: CalibPower65(j)=mean (ChannelPower65(j, k));
   Finally convert this power to dB:
CalibLevel65 dB(j)=10*log 10(CalibPower65(j)); for j=0 . . . 3;
   Note that this 10*log 10( ) contains an implicit sqrt( ) to convert from CalibPower to CalibMagnitude.
   Although insertion gains and CR are selected for each individual user, other parameters may not, and are defined so as to give good audio quality.

### These are:

(a) Channel compression thresholds, Chan_dBthr, which are expressed as a decibel number relative to the channel level when carrying 65 dB speech-shaped noise, Chan0dBGn_lvl. Chan_dBthr ranges from 0 to −15.
(b) Attack and release times for the channel compressors: att and rel, expressed in milliseconds, the speed with which the compressor responds to changes in input level. Attack times (when the signal level is rising) are usually much less than the release times (when the signal is falling in level), but at least a 2:1 ratio.
(c) The relative level at which a channel compression limiter cuts in above the output of the channel compressor, deltaFSdB, expressed in deciBels, typical values 10-20.
(d) Attack and release times for the channel limiters: t_att_lim and t_rel_lim. These are typically set to 3 and 80 msec respectively.
(C) At the very start of the processing, the following calculations may be completed for each channel (assume that each variable may be calculated on a per channel basis)

### (C.1) Expon=(1−CR)/CR

[CR] may never be below 1.
(C.2) The compression threshold, expressed in dB, is converted to a linear value
cthresh=10\hat{ }(0.05*Chan_dBthr)
   (C.3) A channel calibration factor is calculated. This is referenced to the channel level when carrying 65 dB speech, hence why this was calculated in sect B above.
G0dB_norm=(10\hat{ }(−0.05*CalibLeve165 dB))\hat{ }Expon
   (C.4) Constants are calculated to implement the attack and release times of the system used to calculate the short-term mean level, I. These times are defined as the time for the gain signal to settle to within 3 dB of final value (attacking), or 4 dB of final value (releasing) when a 35 dB step change in level has been applied at the input to the compressor (the numbers 35, 3 & 4 will appear below). For very low values of CR, typically around <1.2, the full gain change is barely above 3 or 4 dB, meanings errors can occur in calculations. Therefore error checking is implemented, requiring the compressor to implement at least this gain change. Calculations of the short-term mean level, I, are updated every frame, using a calculated sampling rate, which depends on the FFT size, degree of overlap and sample-based sampling rate.
FsFFT=Fs/(FFTsize/Overlap)=16000/(128/2)=250;
   Frames per seconds are calculated. The overlap between FFTframes is 50%, hence “/2” figure.

### Calculate:

(i) min_dstpdB=35/8;
To ensure that no problems at low CRs. Here the value used is to divide by 8 to get greater than 4 dB change, effective when CR<=1.14
(ii) dstp_att=max(min_dstpdB, 35−3*CR/(CR−1));
Select maximum gain change value.
(iii) dstp_rel=max(min_dstpdB, 35−4*CR/(CR−1));
Select maximum gain change value.
(iv) k_att=10\hat{ }(0.05*(−dstp_att/(t_att*FsFFT/1000)));
t_att is converted to milliseconds
(v) k_rel=10\hat{ }(0.05*(−dstp_rel/(t_rel*FsFFT/1000)));
(C.5) Constants may be calculated to implement the attack and release times of the compression limiter guarding each channel from overload.

### (i) CRlim=100;

Very high CR so as to get true limiter
(ii) dstp_att=max(min_dstpdB, 35−3*CRlim/(CRlim−1));
dstp_rel=max(min_dstpdB, 35−4*CRlim/(CRlim−1));
   (iii) k_att=10\hat{ }(0.05*(−dstp_att/(t_att_lim*FsFFT/1000)));
k_rel=10\hat{ }(0.05*(−dstp_rel/(t_rel_lim*FsFFT/1000)));

### (iv) ExponLim=(1−CRlim)/CRlim;

(v) deltaFSlin=10\hat{ }(−0.05*deltaFSdB);
The difference ratio between channel compressor action and limiter action.
(C.6) Initialise “state” vectors that will carry the most recent versions of the channel mean levels.
for j=1, 2, 3, 4
ChanMeans(j)=Cthresh(j);
ChanLimMeans=Cthresh(j);
End
   (D) Frame-based processing
   For every FFT frame an array is expected (xf) of frequency-domain samples. Apart from the FFT array to process, and the pre-calculated constants (insertion gains, compressor settings, calibration constants), a “state” vector of the running means of the channel compressors may be passed in and the channel limiters.

### Pseudocode

function [xfproc, ChanMeans, ChanLimMeans]=implement_hearing_aid(xf, ChanMeans, ChanLimMeans);
   Which comprises the following steps:
   (D.1) Implement linear insertion gains
xf_ig(i)=xf(i) Glin(i)
   (D.2) Calculate compressor channel powers in a similar method used for calculating channel levels in calibration:
   (i) for j=1, 2, 3; ChannelPower65(j)=0;
   Initialise array. This is only needed at very start.
   (ii) Apply insertion gain to xf:
xf_ig(i)=xf(i)*Glin(i);
   (iii) Calculate the power in each FFT “bin”
BinPower(i)=xf_ig(i)·*conj(xf_ig(i);
   (iv) Sum the powers from each bin into its relevant compression channel. Start and end bins are given above, in variables ChanjFFTbinStart to ChanjFFTbinEnd
for j=1, 2, 3, 4
ChannelPower(j)=sum(BinPower(i)); (NB T spans several bins)
ChannelLevel(j)=sqrt(ChannelPower(j));
end
   In the calculation look the sqrt( ) function is computationally heavy.
   (D.3) 4 gains may be calculated, one for each compression channel. Therefore, a running average is generated. If the new signal level is higher than the previously measured mean level, then the signal is deemed to be “attacking”. If the signal is deemed ‘attacking’, the faster attack time constants are used. If the new signal level is less than or equal to the previously measured mean level, then the signal is deemed to be “releasing”. If the signal is deemed ‘releasing’, the slower release time constants are used. The max( ) function is used to stop the NewChanMeans dropping below the compression threshold. If this is not implemented, then after a long period of silence, if a high level is experienced, the compressor may take a long time to come out of a very low mean level.
   (i) Generate new mean values for both a channel compressor and its limiter
for j=1, 2, 3, 4
   Calculate new ChannelMean for compressor
if ChannelLevel(j)>ChanMeans(j)
k=k_att;
else
k=k_rel;
end
NewChanMeans(j)=max(cthresh(j), (1−k).*ChannelLevel(j)+k.*ChanMeans);
   The limiter value is calculated in a similar way to the mean calculation, its mean value tracks relative to the compressor levels
LimiterLevel(j)=ChanLevel(j)*deltaFSlin(j);
if LimiterLevel(j)>ChanLimMeans(j)
k=k_attlim; %% in FFT implementation this may be unity.
else
k=k_rellim;
end
NewLimMeans(j)=max(cthresh(j), (1−k).*LimiterLevel(j)+k.*ChanLimMeans(j));
end
   (ii) Calculate compressor gain from new mean level, but also, in some embodiments, add in an extra gain reduction based on the ratio of the limiter mean to the compressor mean. The computational complexity of (a) a divide and (b) two exponentiations, may be removed using look up tables to eliminate exponentiations.
Gain(j)=(NewChanMeans(j) \hat{ }Expon(j))*G0dB_norm(j);
if NewChanMeans(j)<NewLimMeans(j)//Limiter will cut in.
Gain(j)=Gain(j)*(NewLimMeans(j)/NewChanMeans(j)) \hat{ }ExponLim(j));
end
   (iii) Expand the 4 channel gains up to the FFT array size. Each gain is assigned to the bin index from which the corresponding channel power was calculated. Indexes were stored in variables ChanjFFTbinStart to ChanjFFTbinEnd
   Initialise array once, at the start of processing.
GainFFT=zeros(1,NFFT);
   Then in every frame (and account for negative frequencies in filling FFT array, if necessary):
for j=1, 2, 3, 4
GainFFT(ChanjFFTbinStart(j) . . . ChanjFFTbinEndChannelPower(j)=Gain(j);
End
   (iv) This leaves GainFFT as an array with rectangular steps at the channel edges. This could cause errors when the values are transformed back into the time domain. Therefore the edge values are smoothed with a 3-tap FIR filter, whose coefficients are Tap3=[0.28 0.44 0.28], which is indexed by k. The filter is “run” forwards & backwards across the entire half of the (frequency domain) array, taking care to ensure that the filtering does not “shift” the Gain function relative to its starting points. Since it is a symmetric FIR filter, forwards and backwards are the same, meaning the same code can be applied for the second pass, but with a different starting array.
   (iv.1) Pass 1: Remove potential overlap/indexing problems at the ends of the arrays.
for i={0, 63}
SmootheGain1(i)=Gain (i);
end
   Perform an FIR filter on the edge values
for i=2 . . . 62
SmootheGain1(i)=Gain(i−1)*Tap3(1)+Gain(i)*Tap3(2)+Gain(i+1)*Tap3(3);
end
   (iv.2) Pass 2: Remove the potential overlap/indexing problems at the ends of the arrays.
for i={0, 63}
SmootheGain2(i)=SmootheGain1 (i);
end
   Perform an FIR filter on the edge values
for i=2 . . . 62
SmootheGain2(i)=SmootheGain1(i−1)*Tap3(1)+SmootheGain1(i)*Tap3(2)+SmootheGain1(i+1)*Tap3(3);
end
   (iv.3) Expand SmootheGain2 array back out to negative frequencies, if necessary.
   (iv.5) Apply compressor gain to array which has already had the insertion gain applied.
for i=0 . . . 63
xf_proc(i)=xf_ig*SmootheGain2(i);
end
   (iv.5) Update and save variables holding these mean levels
ChanMeans=NewChanMeans;//4 channels
ChanLimMeans=NewLimMeans;//4 channels
   (iv.6) return xf_proc from function, along with updated means (or keep them safe until next frame)
   (V) Perform an inverse FFT on this data frame.
   (i) Pseudocode: xproc=inv_fft(xf);
   Unless using an audio-specific inverse FFT function, the output of this function should be real. If the output is returned as an array of complex numbers, then a check may be performed during development to ensure that the imaginary parts are zero.
   Once the checks have been performed, discard the imaginary part and keep the real part. Additionally, if the forward and backward fft( ) functions are reciprocal, there should be no change in scaling of the audio.
   (ii) Perform the same point-by-point multiplication, as in the windowing function described in section (5) above.
   Pseudocode: for i=0 . . . 127
xwproc(i)=W(i)*xproc(i);
   (VI) Perform insertion of new frame of data into output audio stream
   The earliest 64 samples of xwproc (0 . . . 63) are overlapped with the last 64 samples of the previous frame of xwproc and added together and indexed as the next available time buffer to be sent to the output stream (prepared for once the output stream has finished playing out the last output buffer).
   This is called an “overlap-add” procedure. The latter 64 samples from xwproc are saved for the arrival of the next version of xwproc.
   (i) Pseudocode output16(i)=xwproc(i)+xwproc′(i+64); for i=0 . . . 63
xwproc′=xwproc; II save for next iteration of algorithm.
   where xwproc′ is the previously calculated frame.
   “output16” is therefore a 64-long array of audio samples, at the 16 kHz sampling rate.
   (ii) In embodiments, if the original audio sampling rate was 8 kHz, an output buffer is created consisting of the odd-numbered elements of output16. No low-pass filtering is necessary since there should be no alias components due to the low-pass filter performed at stage III(6.1).
   Pseudocode: output8=output16(1, 3, 5, . . . 63);

In embodiments, if the original audio sampling rate was 16 kHz, the output buffer is the same as output16.

So overall, the frame-based processing takes an input buffer (size of 32 samples at 8 kHz or 64 samples at 16 kHz) and produces one output buffer (size of 32 samples at 8 kHz or 64 samples at 16 kHz), thus maintaining a constant flow of audio between input and output.

The double-windowing function, with the overlap-add, produces unity recombination where the inverse-fft output arrays overlap. If a “buzz” at the framerate appears in the output audio then a possible error has occurred.

According to some embodiments the user of the user device, or a network operator, can selectively activate or deactivate a setting which provides the audio signal modification. This may be useful, for example, if a user does not require the audio modification for some reason. This may also be useful where the user device of the user is also used by other people who may not require the audio modification.

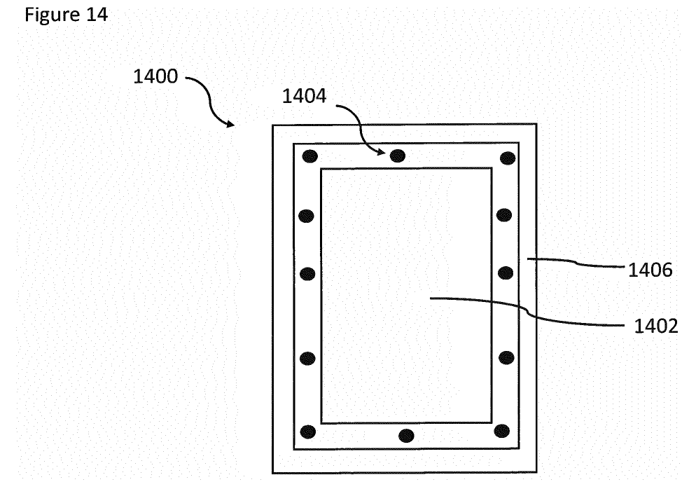

A further aspect is shown in FIG. 14, which shows a user device 1400. The user device 1400 may be a mobile phone, for example, or indeed any other kind of digital device. The user device 1400 comprises a display 1402. The user device 1400 also comprises a plurality of microphones, as represented by the black circles 1404. In this example the device comprises twelve microphones. It will be understood that in other examples more or fewer microphones may be provided. Such a user device may operate in conjunction with the earlier described embodiments. The array of microphones 1404 can receive noise, and transmit information of that noise, to the network to be processed as previously described. The microphones 1404 may be directionally focused. The microphones may be linked to an operating system of the user device 1400. In turn, the operating system may be communicatively linked to the hearing profile of the user, which enables audio signal adjustment unique to that person. By way of example the user device 1400 may be placed at the front of a desk or on a support, and picks up audio signals (e.g. voice or music). Those audio signals can then be transmitted by the user device 1400 to the network, where they may be processed for tailoring audio signals to the user of the user device, in conjunction with the hearing profile for that user.

The user device 1400 further comprises a coating or layer 1406. The coating 1406 may be in the form of a metal band or a coil. The coating 1406 may act as an antenna and/or an induction loop and/or a T-coil (tele-coil), or indeed any other assistive device or accessory to communicate from the user device 1400 to a hearing aid of a user. The coating 1406 may further comprise a battery and/or processor and/or memory, so as to increase battery life and/or processing power and/or storage capability of the user device 1400. This can also help the T-coil or other applications needed to connect to hearing aids. The coating 1406 may also have tagging and/or internet of things (IoT) capability incorporated therein. Such capability may specify a user's unique Hearing Identification Code. In some embodiments the coating 1406 is in the form of a casing which is attachable and detachable from the user device 1400.

Accordingly, improved audio enhancement is provided tailored for the hearing requirements of a particular user in a real time manner based on and specific to pre-measured and configured hearing loss and needs of the individual.

The described methods may be implemented by a computer program. The computer program which may be in the form of a web application or ‘app’ comprises computer-executable instructions or code arranged to instruct or cause a computer or processor to perform one or more functions of the described methods. The computer program may be provided to an apparatus, such as a computer, on a computer readable medium or computer program product. The computer readable medium or computer program product may comprise non-transitory media such as as semiconductor or solid state memory, magnetic tape, a removable computer memory stick or diskette, a random access memory (RAM), a read-only memory (ROM), a rigid magnetic disc, and an optical disk, such as a CD-ROM, CD-R/W, DVD or Blu-ray. The computer readable medium or computer program product may comprise a transmission signal or medium for data transmission, for example for downloading the computer program over the Internet.

An apparatus or device such as a computer may be configured to perform one or more functions of the described methods. The apparatus or device may comprise a mobile phone, tablet, laptop or other processing device. The apparatus or device may take the form of a data processing system. The data processing system may be a distributed system. For example, the data processing system may be distributed across a network or through dedicated local connections.

The apparatus or device typically comprises at least one memory for storing the computer-executable instructions and at least one processor for performing the computer-executable instructions.

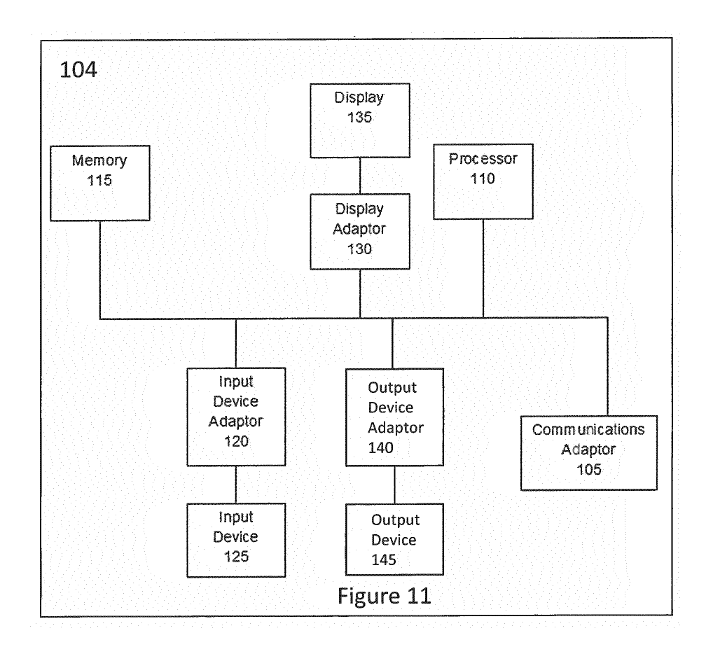

FIG. 11 shows the architecture of an example apparatus or device 104. The apparatus or device 104 comprises a processor 110, a memory 115, and a display 135. These are connected to a central bus structure, the display 135 being connected via a display adaptor 130. The example apparatus or device 100 also comprises an input device 125 (such as a mouse, audio input device and/or keyboard), an output device 145 (for example an audio output device such as a speaker or headphone socket) and a communications adaptor 105 for connecting the apparatus or device to other apparatuses, devices or networks. The input device 125, output device 145 and communications adaptor 105 are also connected to the central bus structure, the input device 125 being connected via an input device adaptor 120, and the output device 145 being connected via an output device adaptor 140.

In operation the processor 110 can execute computer-executable instructions stored in the memory 115 and the results of the processing can be displayed to a user on the display 135. User inputs for controlling the operation of the computer may be received via input device(s) 125.

1. A method comprising:
conducting a hearing test for a user over a communication link established between a network entity in a communication network and a user device of a user;
wherein the hearing test comprises providing audio stimuli to the user device at a plurality of test frequencies over the communication link, the audio stimuli comprising bands of white noise less than an octave wide and based on one or more human voices, and monitoring responsiveness to the audio stimuli received from the user device;
generating a hearing profile based on results of the hearing test; and
storing the hearing profile and information associated with the user in a memory of a network entity, such that the hearing profile is available for modifying of audio signals to the user device.

2. The method as set forth in claim 1, wherein the information associated with the user comprises at least one of an identifier of the user and an identifier of the user device.

3. (canceled)

4. The method as set forth in claim 1, wherein the audio stimuli comprises ⅓ octave wide bands of white noise.

5. The method as set forth in claim 1, wherein the providing audio stimuli to the user at a plurality of test frequencies comprises providing audio stimuli at two or more of 500 Hz; 1000 Hz; 2000 Hz; 3000 Hz; 6000 Hz.

6. The method as set forth in claim 1, comprising obtaining an indication of hearing loss of the user, and using the indication of hearing loss to determine an initial volume of the hearing test.

7. The method as set forth in claim 1, comprising adjusting a volume of the audio stimuli at each test frequency in response to the monitoring responsiveness, wherein, in response to a positive response from the user the method comprises decreasing the volume of the audio stimuli; and wherein, in response to a null response from the user, the method comprises increasing the volume of the audio stimuli.

8. (canceled)

9. (canceled)

10. The method as set forth in claim 1, wherein a duration of each audio stimuli is at or about 1000 ms.

11. The method as set forth in claim 1, wherein each audio stimuli includes one or more ramps of increasing/decreasing volume between a background noise level and 60 dB or about 60 dB.

12. (canceled)

13. The method as set forth in claim 1, comprising using the stored hearing profile of the user to modify audio signals to the user in real-time, the modifying of the audio signals being carried out at the network entity such that modified audio signals are delivered to the user device of the user.

14. The method as set forth in claim 13, wherein the modifying audio signals comprises one or more of: filtering the audio signal; adjusting an amplitude of the audio signal; adjusting a frequency of the audio signal; adjusting a pitch of the audio signal.

15. The method as set forth in claim 13, wherein the modifying audio signals comprises modifying voice signals of a second user in a call between the user and second user.

16. The method as set forth in claim 13, comprising: enabling selective activation or deactivation of a setting which provides the audio signal modification.

17. The method as set forth in claim 13, comprising measuring ambient noise using one or more microphones of the user device, receiving ambient noise information from the user device at the network entity that has the communication link with the user device, and storing the received ambient noise information at the network entity which stores the hearing profile for use in modification of audio signals to the user.

18. The method as set forth in claim 13, comprising determining a channel insertion gain for delivering the audio signals to the user device; wherein the channel insertion gain is applied prior to dynamic compression of the audio signals to the user.

19. The method as set forth in claim 13, comprising splitting the audio signals in to multiple channels, and determining a power level for each channel.

20. (canceled)

21. (canceled)

22. A method comprising:
participating in a hearing test for a user over a communication link established between a user device and a network entity in a communications network to provide a hearing profile for a user;
wherein the hearing test comprises receiving audio stimuli at the user device at a plurality of test frequencies over the communication link, the audio stimuli comprising bands of white noise less than an octave wide and based on one or more human voices, and providing one or more responses to the audio stimuli to the network entity; and
subsequently receiving audio signals at the user device modified in dependence on the hearing profile.

23. A server arranged to carry out the method of claim 1.

24. A user device arranged to carry out the method of claim 22.

25. A computer-readable medium comprising instructions that when executed, cause a processor to carry out the method of claim 1.

26. A computer-readable medium comprising instructions that when executed, cause a processor to carry out the method of claim 22.
